(19) United States
(12) Patent Application Publication
(10) Pub. No.: US 2019/0044336 A1
(43) Pub. Date: Feb. 7, 2019

(54) A MULTIFUNCTIONAL POWER DISTRIBUTION APPARATUS

(71) Applicant: KONINKLIJKE PHILIPS N.V., EINDHOVEN (NL)

(72) Inventors: BERNHARD WAGNER, EINDHOVEN (NL); PETER LUERKENS, EINNDHOVEN (NL); AXEL SEMKE, EINDHOVEN (US)

(21) Appl. No.: 16/074,715

(22) PCT Filed: Feb. 1, 2017
(86) PCT No.: PCT/EP2017/052182

§ 371 Date: Aug. 1, 2018

(30) Foreign Application Priority Data
Feb. 2, 2016 (EP) 16153857.4

(51) Int. Cl. H02J 3/32; H02J 7/00; H02J 9/06; A61B 6/00

(52) U.S. Cl. CPC H02J 3/32; A61B 6/56; H02J 9/061; H02J 7/0026

(57) ABSTRACT

Power supplies for supplying medical systems in hospitals must be designed to accommodate a demanding range of requirements. The instantaneous power demand from modern CT systems can reach hundreds of kilo Watts. Dimensioning a hospital utility power system to provide this instantaneous power level is expensive. The usage pattern of medical systems in hospitals means that the instantaneous power is required only for a low duty cycle, with an average power demand of such a system being at least one order of magnitude lower. Therefore, the present application proposes a multifunctional power distribution system, with a charging mode, an operation mode, a backup mode, and a bypass mode. In the operating mode, the average power level may be supplied from the utility mains, but the relatively infrequent peak power demands may be provided from an electrical energy storage element, which is charged by the utility mains supply.

### FIELD OF THE INVENTION

This invention relates to a multifunctional power distribution apparatus, a medical equipment system, a method for controlling a multifunctional power distribution apparatus, a computer program element, and a computer-readable medium.

### BACKGROUND OF THE INVENTION

Medical equipment systems comprising medical imaging equipment, such as X-ray or CT scanners, characteristically have equipment which requires a high level of pulsed power, or a lower level of continuous power. For example, in the area of angiographic imaging, this characteristic is caused by the production of X-ray pulses according to a desired frame rate of an angiography sequence. If pulses of high power having a small duty cycle are created, a large ratio of peak power to average power results. On the other hand, some consumers of power in the imaging system may consume power continuously, but at a much lower magnitude.

The provision of power distribution systems sized for a peak power requirement of a medical imaging system is expensive. Conventionally, the utility mains supply must be rated for the peak power, even though the peak power level is commonly only reached for short time durations. US 2008/0112537 discusses a power storage device configured to share power delivery with an input power line in order to reduce peak load requirements of the input power line. Such systems can, however, be further improved.

### SUMMARY OF THE INVENTION

It would, thus, be advantageous to have a technique for providing an improved power distribution apparatus for powering medical equipment.

The object of the present invention is solved by the subject-matter of the independent claims, wherein further embodiments are incorporated in the dependent claims.

These, and other aspects of the present invention will become apparent from, and be elucidated with reference to, the embodiments described hereinafter.

According to a first aspect of the invention, there is provided a multifunctional power distribution apparatus. The apparatus comprises:

input terminals enabling connection of the apparatus to a source of electrical energy, a charging unit connected to the input terminals;

an electrical energy storage element configured to receive electrical energy from the charging unit;

DC load terminals configured to supply electrical energy to a load, a power switching network enabling an adaptable configuration of the charging unit, the electrical energy storage element, and the DC load terminals; and

a control unit configured to control the charging unit and the power switching network.

The control unit is configured to set the power switching network into at least one of the following modes: (i) a charging mode in which the electrical energy storage element is charged by the charging unit, (ii) an operating mode in which electrical energy is supplied to the DC load terminals from the electrical energy storage element and the charging unit, and the electrical energy storage element can be charged, (iii) a backup mode in which electrical energy is supplied to the DC load terminals exclusively from the electrical energy storage element, and (iv) a bypass mode in which electrical energy is provided to the DC load terminals exclusively from the charging unit.

Therefore, a flexible power supply system is provided. The storage of electrical energy in the electrical energy storage element enables components in the utility mains side of the multifunctional power distribution apparatus to be rated closer to the average load power specified for the multifunctional power distribution apparatus, rather than the peak power required by the multifunctional power distribution apparatus.

Therefore, components on the utility-mains side of the multifunctional power distribution apparatus can be reduced in cost. The utility mains does not experience sudden spikes in power usage, because instantaneous peak power demands are drawn from the electrical energy storage element. The multifunctional power distribution apparatus can function entirely in a backup mode, providing an uninterruptible power supply to the DC load terminals in the event of a power supply failure.

Accordingly, the multifunctional power distribution apparatus may also bypass the electrical energy storage element, for example in a fault condition of the electrical energy storage element.

According to an embodiment of the first aspect, a power distribution apparatus according to the first aspect is provided, wherein the charging unit is configured to charge the electrical energy storage element using (i) an adjustable DC current or (ii) an adjustable DC voltage or (iii) according to a predefined charging curve or (iv) according to a predefined charging characteristic.

Therefore, the electrical energy storage element can be charged by different profiles, using either an adjustable current or an adjustable voltage profile.

According to an embodiment of the first aspect, the power distribution apparatus is provided, wherein the electrical energy storage element comprises a positive-side electrical energy storage element, and a negative-side electrical energy storage element, which are both connected to a protective earth node.

Accordingly, the multifunctional power distribution technique can be applied to a dual-rail voltage supply, also known as a DC-link voltage circuit.

According to an embodiment of the first aspect, the power distribution apparatus is provided, further comprising a current sensor configured to monitor a differential current flowing between the positive-side electrical energy storage element and the protective earth node. The control unit is configured to adjust a set point of the charging unit, in order to minimize the differential current between the positive and negative side electrical energy storage elements.

Accordingly, a charge imbalance between electrical energy storage elements on the positive-rail and negative-rail side of the power distribution apparatus may be identified. Upon correcting the imbalances, a symmetric dual-rail DC voltage supply can be provided.

According to an embodiment of the first aspect, a power distribution apparatus is provided, further comprising:

an electrical energy storage element management system.

The electrical energy storage element comprises a plurality of cells, and the electrical energy storage element management system is configured to supervise cells of the plurality of cells of the electrical energy storage element, to detect an undesired state between cells of the electrical energy storage element, and to compensate for the undesired state.

Accordingly, the power distribution apparatus can identify faults occurring with individual cells, or groups of cells, of an electrical energy storage element, and address the faults automatically.

According to an embodiment of the first aspect, the power distribution apparatus is provided, wherein the charging unit is configured to provide an average power level of an expected load characteristic at the charging unit load terminals.

Accordingly, the charging unit may be de-rated, to enable a reduction in component cost. However, the electrical energy storage unit can still be charged over time to provide the peak power requirement of a medical system connected to the electrical energy storage element.

According to an aspect of the first embodiment, the power distribution apparatus is provided, wherein, between the charging mode and the operation mode, the control unit is further configured to set the power switching network into a transition mode. In the transition mode, the power switching network is configured to connect a resistance in series between the electrical energy storage element and the DC load terminals, to prevent the occurrence of an inrush current.

Accordingly, when the power distribution apparatus is connected to an item of equipment with large input storage capacitors, and the mode is changed from the charging mode to the operation mode, damage to the power distribution apparatus can be avoided.

According to an embodiment of the first aspect, the power distribution apparatus is provided, further comprising:

a charge level detector configured to obtain a charge level of the electrical energy storage element. The control unit is further configured to compute a remaining operating time, such as a share, such as a percentage of the residual charge or energy, of equipment connected to the multifunctional power distribution apparatus based on the charge level of the electrical energy storage element.

Accordingly, during the backup mode, it is possible to provide feedback to a medical professional using equipment connected to the power distribution apparatus about the amount of time remaining during a power fault. In the event of a power failure during a catheterization or other interventional operation, this could enable a safer emergency conclusion of the procedure.

According to an embodiment of the first aspect, the power distribution apparatus is provided, wherein the power switching network comprises a first switching element configurable to connect the electrical energy storage element to the DC load terminals, a second switching element configurable to connect the output of the charging unit to the electrical energy storage element, and a third switching element configurable to connect the output of the charging unit directly to the DC load terminals.

Accordingly, the power distribution apparatus may be configurable into a plurality of modes.

According to an embodiment of the first aspect, a power distribution apparatus is provided, wherein the apparatus is further configured to prevent the occurrence of a switching event in the path between the electrical energy storage element and the DC load terminals during a transition between the operating mode and the backup mode. Accordingly, power “spikes” caused by the transition between the operating mode and the backup mode will be significantly reduced or removed. Some medical equipment is sensitive even to very small power supply fluctuations, which are prevented according to this embodiment.

According to a second aspect of the invention, a medical equipment system is provided. The medical equipment system comprises:

a medical imaging apparatus, and

the multifunctional power distribution apparatus of the first aspect, or its embodiments, as described above.

The input terminals of the multifunctional power distribution apparatus are connectable to a utility power supply, and the DC load terminals of the multifunctional power distribution apparatus is configured to supply electrical energy to the medical imaging apparatus as a load.

Accordingly, in the medical equipment system, many power supply components may be removed, or at least de-rated, because they are only required to supply to the multifunctional power distribution apparatus the average power, and not the peak load power, demanded by the medical equipment system.

According to a third aspect of the invention, there is provided a method for controlling a multifunctional power distribution apparatus, comprising:

a) charging the electrical energy storage element using the charging unit;
b) monitoring, using the control unit of the multifunctional power distribution apparatus, a power demand requirement of a load connected to the DC load terminals of the multifunctional power distribution apparatus using the control unit;
c) computing a configuration of the power switching network using the power demand requirement of the load;
d) configuring the power switching network into one of (i) a charging mode, (ii) an operating mode, (iii) a backup mode, and (iv) a bypass mode.

According to the method, a multifunctional power distribution apparatus can function to supply power to a medical application directly from the utility mains, from a combination of an electrical energy storage element and the utility mains, or in a backup mode, entirely from an energy storage element. Thus, a flexible power supply method is provided. In addition, the method only demands the supply of an average power level, rather than the potential peak power level of a system connected to the multifunctional power distribution apparatus.

According to an embodiment of the third aspect, in step d), the power switching network is further configurable into (iv) a bypass mode.

According to an embodiment of the third aspect, the method is provided, further comprising:
a1) detecting a fault condition of the source of electrical energy at the input terminals;
d1) configuring the power switching network into the backup mode; further comprising step e):
e) supplying electrical energy to the load exclusively from the electrical energy storage element.

According to a fourth aspect of the invention, a computer program element for controlling an apparatus according to one of the first aspect or embodiment is provided, which, when the computer program element is executed by a control unit, is adapted to perform the steps of one of the third aspect or its embodiments.

According to a fifth aspect of the invention, there is provided a computer-readable medium having stored the computer program element of the fourth aspect.

In the following description, the term “electrical energy storage element” means a circuit component capable of storing energy, such as a capacitor, a double layer capacitor, or a super capacitor, or a battery, such as a stack of lithium ion batteries, for example.

In the following description, the term “power switching network” means a plurality of switching means, and associated interconnections, capable of redirecting current in a power distribution apparatus. The switching means may be electro-magnetically actuated contactors, or semiconductor switching means such as power transistors. The switching means may be controlled by the control unit to configure the power switching network into one of a plurality of states enabling different functional modes of the multifunctional power switching network to be provided.

Accordingly, a basic idea of the technique discussed is to provide a system supply architecture for a medical equipment system which overcomes the drawbacks of power distribution systems supported by uninterruptible power supplies. Full performance up to the collective rated power of all connected consuming elements can be realized at a significantly reduced level of component and installation costs.

### BRIEF DESCRIPTION OF THE DRAWINGS

Exemplary embodiments will be described with reference to the following drawings:

FIG. 1 shows a system configured for medical imaging according the second aspect.

FIG. 2 shows an example of a prior-art technique for supplying electrical energy to a medical equipment system.

FIG. 3 shows a some examples of a power usage characteristic of a typical medical equipment system operated in a random sequence over time.

FIG. 4 shows a multifunctional power distribution apparatus according to the first aspect.

FIG. 5 shows a system architecture of a multifunctional power distribution apparatus connected to a variety of consumers.

FIG. 6 shows an electrical circuit schematic of a double-layer capacitor implementation of a multifunctional power distribution system.

FIG. 7 shows an alternative electrical circuit to that of FIG. 6, with an alternative output architecture.

FIG. 8 shows an electrical circuit schematic of a single-sided implementation of a multifunctional power distribution system.

FIG. 9 shows a method according to the third aspect.

### DETAILED DESCRIPTION OF EMBODIMENTS

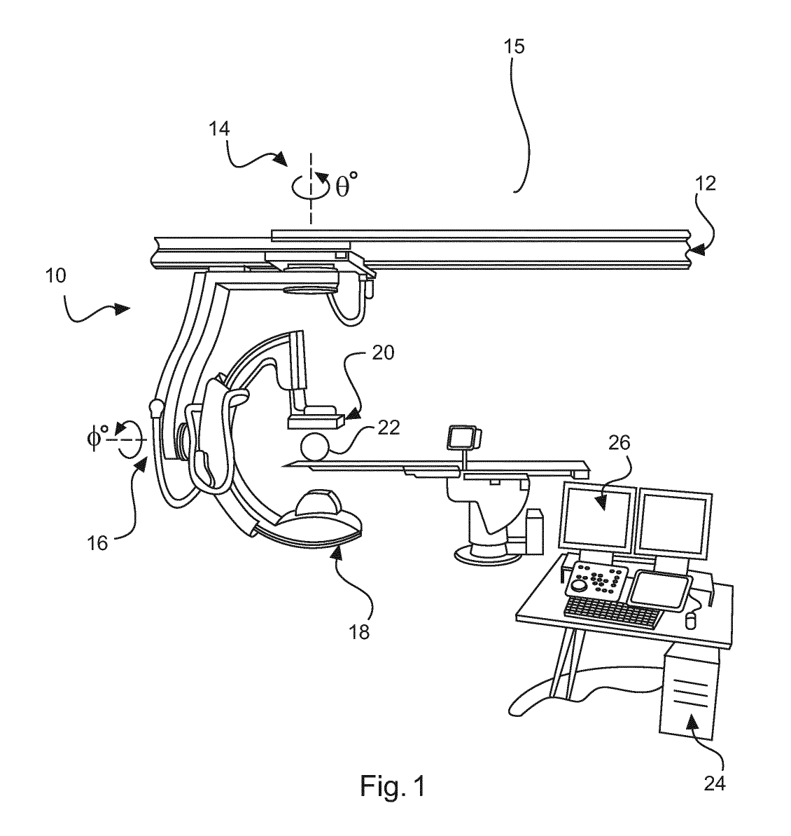

FIG. 1 shows a catheterization laboratory (medical equipment system 15) of a hospital containing elements of medical imaging equipment which can commonly be found in such laboratories. In the catheterization laboratory, there is a C-arm imaging system (medical imaging apparatus) 10 suspended from the ceiling 12 of the catheterization laboratory. The C-arm comprises a first rotation bearing 14 enabling the entire C-arm to be rotated around an azimuth angle θ°, and a second rotation bearing 16 enabling the C-arm's head to be tilted through an elevation angle φ°. The C-arm's imaging head comprises an X-ray emitter 18 and an X-ray detector 20. In operation, the C-arm is positioned with the X-ray emitter 18 disposed to emit an X-ray beam through a region of interest 22, so that the X-ray detector 20 provides an X-ray image of the region of interest 22. Typically, other electrically powered items are present in the room, such as a control computer 24 and imaging display 26. Other items of equipment (not shown) which could also be used comprise items such as vital-signs monitoring equipment, ultrasound imaging equipment, and ancillary electrical equipment such as ventilation fans, for example. Such a medical equipment system has widely-varying power supply needs.

An analysis of all consumers of electrical power in such a medical equipment system demonstrates that there are two basic groups of consumers. One consumer group comprises consumers which continuously draw low or medium power between 100 W, up to a few kW. The computer 24 and imaging display 26 could be considered to be in this category.

A second group of consumers demand a low level of continuous power (for example, 2 kW), whilst having a very high power peak power requirement of up to 150 kW, for example. In this case, the X-ray tube 18 could have such a high peak power requirement when making angiographic imaging sequences, for example. Other high-energy items are magnetic resonance gradient amplifiers, for example. In the case of a traditional X-ray machine, the peak electrical power is consumed for small periods of time (seconds). In the case of an angiography or fluoroscopy imaging setup, the period of elevated power demand could comprise bursts lasting, typically, for thirty minutes. Over this time, the demand could vary with adjustments in the frame rate of the sequence. Typically, the pulse frequencies, and scan duration, may be considered to be random, and dependent on dedicated application parameters relating to a patient's physical figure, and the mode of operation of the equipment.

The total average power within an observed time period T can be calculated according to (1):

P_{avg}=(1/T)*ΣPᵢ*Tᵢ (1)

Pᵢ is the pulse power of a pulse at instantᵢ, and Tᵢ is the pulse duration of the pulse, for i=1 . . . n. T represents the total observed period of time which comprises all instances of Tᵢ, as well as the pauses which occur between the pulses.

The present situation is that even in the case that peak power is demanded by equipment for short periods of time, the hospital utility supply must be dimensioned to provide such a peak demand. In practice, power supply installations in hospitals need to be dimensioned for a consumption at the order of several hundreds of kW, whereas the average power consumed by the equipment may be in a lower order of magnitude.

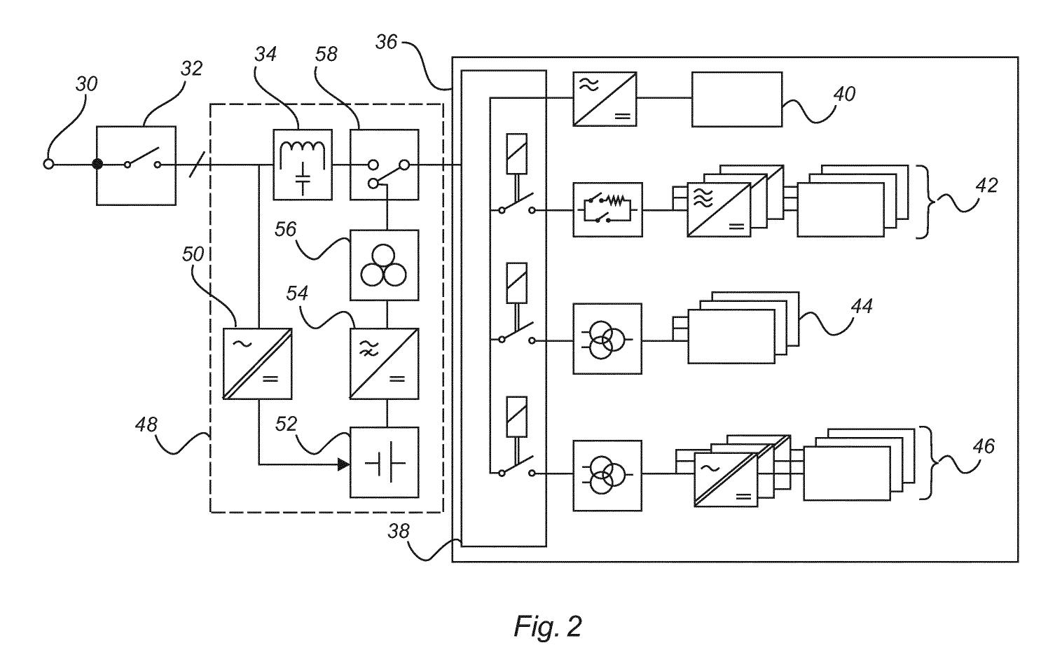

FIG. 2 shows the range of consumers in a typical installed medical system which is permanently connected to a 3-phase hospital mains system continuously transferring power to the system.

In FIG. 2, the hospital mains 30 is provided to a main switch 32. The three-phase power is then connected via a filter 34. The medical system 36 comprises a power distribution unit 38, providing power to various consumer types 40, 42, 44, 46 via a contactor network. Consumer type one 40 is a unit which needs to be continuously supplied, as long as the system is installed, such as a mains power-on circuit, or a temperature controller needed for temperature-sensitive components.

Consumer type two 42 represents a high-voltage DC consumer unit, which can be connected to the uncontrolled rectified mains voltage. Such items could be DC/AC converters, supplying powerful consumers. Alternatively, they could be a high voltage source for X-ray tube sources, or large motor drives, as found in a CT scanner, for example. Consumer type three units 44 may be pumps, or fans, which are supplied using a single or a 3-phase AC voltage.

Consumer type four units 46 represent circuits which consume a low voltage, which usually need to be isolated from mains-connected circuits for safety reasons. These may be printed circuit boards for computing or control circuitry or low or medium power consumption up to some few kilowatts, or voltage-controlled fans, for example.

A state of the art uninterruptible power supply (UPS) system is shown in the dotted line box 48. An AC/DC charger 50 is connected between the three-phase wall input and an electrical energy storage element 52, such as a stack of lithium ion battery cells. The electrical energy storage element 52 is charged by the charger 50. A DC to AC converter 54 is connected between the electrical energy storage element 52 and a three-phase transformer 56. The output of the three-phase transformer is connected to the switch 58.

In a normal operating mode (not shown), the mains switch 58 couples the three-phase mains supply, via a mains filter 34, to supply the power distribution unit 38. At the same time, the battery charger 50 charges the electrical energy storage element 52.

In a utility mains power interruption situation, the mains switch 58 is configured to connect the three-phase transformer into the power supply path, so that the medical system 36 is supplied from charge stored in the electrical energy storage element 52. It is noted that as the changeover of the mains switch 58 is made, the entire medical system 36 experiences a power dropout during both the time period needed to detect the power fail event and the switching phase. The dropout phase may be followed by a sharp power spike. This originates either from the switch changeover time, or from the momentary depletion and subsequent recharge of large capacitances on the load side. Therefore, sensitive consumer systems might not perform reliably during the delay of a few milliseconds or longer, until the connection to the battery path is established, controlled to steady-state operation and thus providing a stable output voltage.

In an emergency situation with no utility mains power, the UPS would need to be dimensioned to provide for the peak power consumption of the medical system 36.

Alternatively, the performance of the medical system 36 would be limited to a reduced level. In practice, this could mean that high peak power systems, such as an X-ray sources in a C-arm, could not be used during a power-down situation. Therefore, uninterruptible power supplies capable of supplying the peak power for full X-ray performance are only installed if this feature is essential for the performance of the medical system.

A time-break due to switching of the utility mains and an electrical energy storage element can be avoided in an architecture in which the battery circuit is permanently connected to the consuming load. However, no power is supplied by the electrical energy storage element 52 during normal operation, because the electrical energy storage element 52 is only charged to a desired level. In the case of a mains fault (a low impedance connection to protective earth, for example), energy is transferred from the electrical energy storage element 52 to the load, and the utility mains is disconnected by the mains switch 58. A problem with this arrangement is that the longer the mains switch 58 delays its disconnection, the longer the DC to AC converter 54 feeds stored energy of the electrical energy storage element 52 back into a low impedance short circuit of the mains, potentially damaging the electrical energy storage element 52.

Another architecture (not illustrated) is that of a permanently connected uninterruptible power supply which continuously transfers the needed power to the system. In this case, critical loads are completely decoupled from the mains. No switching action is necessary in the case of a mains breakdown, and a low-impedance breakdown does not lead to a critical situation of the system, because the system can be decoupled by a controlled rectifier located inside the uninterruptible power supply. A drawback of such a configuration are the higher operation costs due to the continuous power transfer between converters inside the uninterruptible power supply.

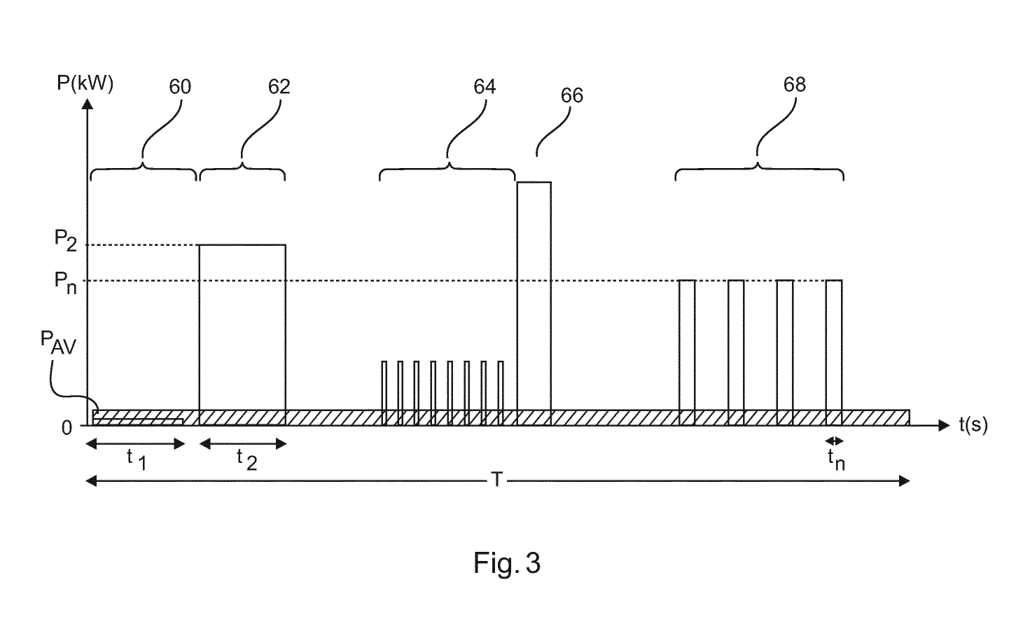

FIG. 3 shows a power use characteristic of a typical medical imaging facility. The y-axis shows power use of an X-ray tube in kW, and the x-axis shows the time in seconds. At region 60 of the graph, a continuous fluoroscopy scan occurs. At region 62, a high power CT scan is made. At region 64, a pulsed fluoroscopy sequence is made. At region 66, a single X-ray exposure is made. At region 68, a multiphase CT scan is performed. The high power CT scan reaches a maximum X-ray tube power of P₂ kW. The multiphase CT scan 68 reaches a maximum X-ray tube power of Pₙ. The average duty cycle 6 defines the ratio of added pulse durations to the total observed period, according to (2):

δ=ΣTᵢ/T (2)

Tᵢ represents the time duration of an X-ray impulse, and T represents the total examination time.

As can be seen from FIG. 3, in common diagnostic X-ray applications, the duty cycle of equipment used during an examination is low. For CT applications, δ is typically lower than 5%. For cardiac applications, δ is typically lower than 3%. For vascular applications, δ is typically lower than 2%. Therefore, the average power of a medical X-ray lab, P_{AV}, is, as shown in FIG. 3, extremely low, compared to the instantaneous requirement of a single X-ray exposure 66, for example. Providing a utility mains supply, and the associated conversion equipment, sized to the peak power requirement of a medical system operating under such duty cycle conditions is expensive, and wasteful. A solution to this problem is presented below.

According to a first aspect, there is provided a multifunctional power distribution apparatus 70.

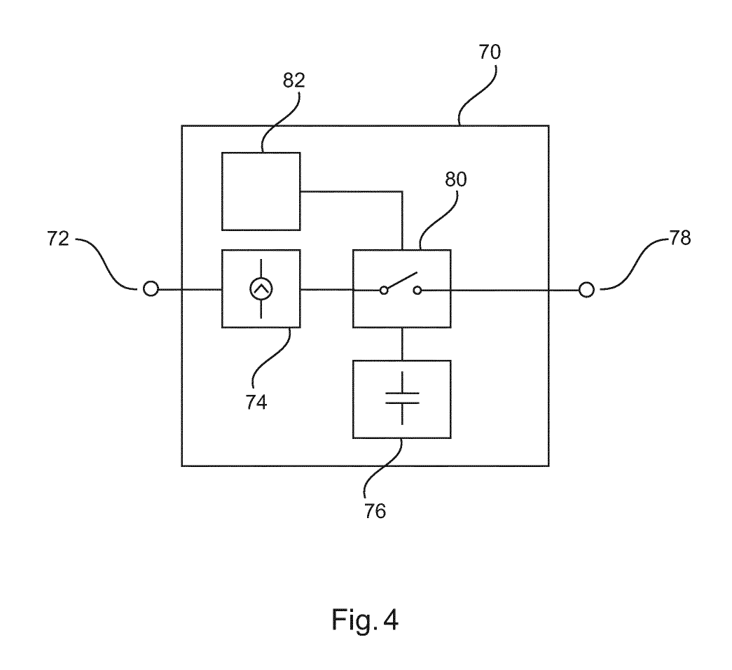

FIG. 4 illustrates a multifunctional power distribution apparatus 70 according to the first aspect.

The apparatus comprises:

input terminals 72 enabling connection of the apparatus to a source of electrical energy,

a charging unit 74 connected to the input terminals,

an electrical energy storage element 76 configured to receive electrical energy from the charging unit,

DC load terminals 78 configured to supply electrical energy to a load,

a power switching network 80 enabling an adaptable configuration of the charging unit, the electrical energy storage element, and the DC load terminals, and a control unit 82 configured to control the charging unit and the power switching network. The control unit 82 is configured to set the power switching network 80 into at least the following modes: (i) a charging mode in which the electrical energy storage element 76 is charged by the charging unit 74, (ii) an operating mode in which electrical energy is supplied to the DC load terminals 78 from the electrical energy storage element 76 and the charging unit 74, and the electrical energy storage element 76 can be charged, (iii) a backup mode in which electrical energy is supplied to the DC load terminals 78 exclusively from the electrical energy storage element 76, and (iv) a bypass mode in which electrical energy is provided to the DC load terminals 78 exclusively from the charging unit 74.

Accordingly, the multifunctional power distribution apparatus may supply a continuous average component of the power demand using a utility mains supply connected to the input terminals 72, but may supply pulsed high-power loads at the peak load power using electrical energy stored in the electrical energy storage element 76. Therefore, components upstream of the multifunctional power distribution apparatus 70 may be resized (de-rated), enabling them to be provided more cheaply. In addition, the utility mains connection of a hospital need not be sized to use the peak power draw of the equipment in the X-ray laboratory, but rather to the average power draw. The charging unit 74 may be rated towards the average power of the load, and not towards the peak power.

Therefore, this system supply architecture overcomes the previously mentioned problems.

According to an embodiment of the first aspect, the charging unit 74 is configured to charge the electrical energy storage element 76 to supply a peak power level of a medical system, whereby the charging unit is also configured to supply an average power level of the medical system to the charging unit load terminals.

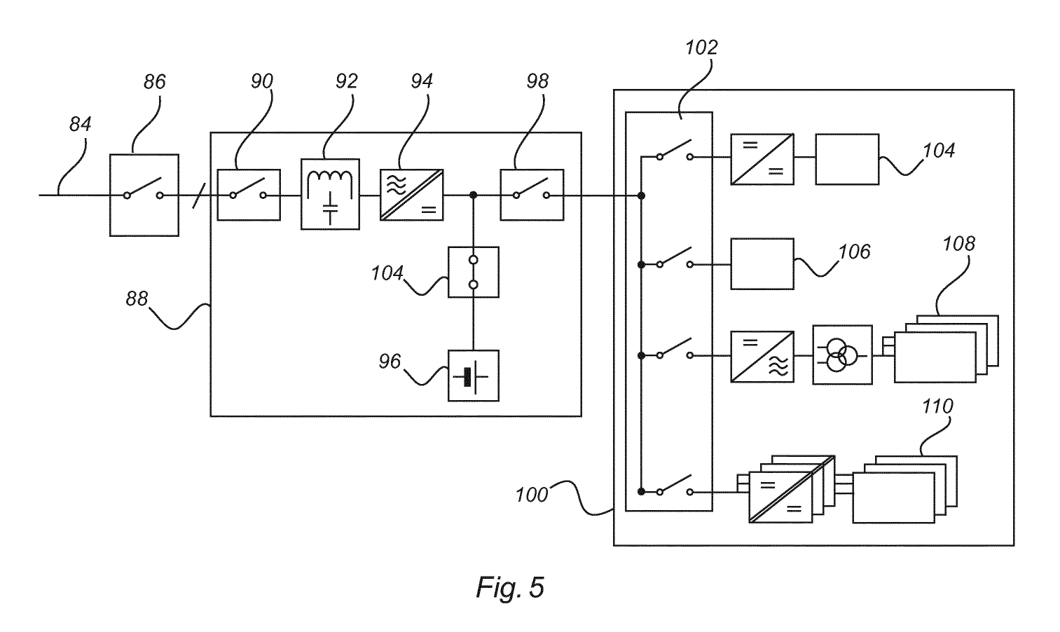

FIG. 5 shows a system architecture for an installed multifunctional power distribution unit according to an embodiment of the first aspect. The voltage and frequency independent uninterruptible power supply 88 is connected to the utility mains connection of the hospital 84 via a wall switch 86. The UPS 88 comprises a mains switch 90, a filter 92, a single phase or a 3-phase charger 94, an electrical energy storage element 96, such as a battery or a super capacitor, and a contactor circuit 98. The voltage and frequency independent UPS 88 therefore stores energy from a hospital's utility mains connection. The network of medical consumer equipment 100 is connected to the voltage and frequency independent UPS 88 via a power distribution unit 102. As discussed previously, the variety of loads may be comprised within the medical system, for example a mains power-on circuit 104, an X-ray high voltage source 106, fans or pumps 108 which are supplied by a single or 3-phase AC voltage, or low voltage circuits 110.

According to an embodiment, the DC energy storage unit 96 (an electrical energy storage element) may comprise batteries, double-layered capacitors, or stacked super capacitors. The electrical energy storage element combines the function of a normal energy supply, as well as an uninterruptible power supply function, for all connected consumers in the entire medical system.

According to an embodiment, the electrical energy storage element 96 can be connected to a DC power bus, which is configured to share electrical energy stored in the electrical energy storage element via all connected consumers using a power distribution unit 102.

Therefore, the electrical energy storage unit can cover peak power loads of the medical system which are much higher than the power which is consumed on average. However, the charging power of the electrical energy storage element 96 only requires the average power. Consumers drawing high peak power pulses with a small duty cycle may be, for example, motors with a high initial starting current, or the high-voltage generating units for X-ray power. These may operate for a duration of several milliseconds, to as a maximum, several tens of seconds.

According to an embodiment, the electrical energy storage element may comprise a set of cells connected in series, in order to provide a total voltage across terminals of the series-connected cells. Additionally or alternatively, cells may be connected in parallel to each other in order to provide the maximum rated current to be consumed by the medical system 100.

According to an embodiment, the cells may be batteries, such as lithium ion cells. Alternatively, the cells may be super capacitor cells, or other cells having the characteristics of a DC voltage buffer, for example electrolytic capacitors.

According to an embodiment, a single phase, or a 3-phase charging unit 94 can be dimensioned to provide the maximum rated average power, preferably with two operation modes for charging: a first operation mode of constant current charging, and a second operation mode of variable current charging with voltage limitation. Other kinds of charging modes are applicable, e.g. following a predefined charging curve or a charging characteristic which may be online calculated by the use of electrical parameters. The nominal charging power may be dimensioned to supply the collective average power consumption of all connected circuits of the entire system.

According to an embodiment, a contactor circuit 98 between the storage 96 and system consumers. The contactor circuit 98 limits inrush currents which are caused by large capacitive loads.

Therefore, the maximum pulse power no longer needs to be transferred from a DC buffer to a 3-phase AC level, and then to be rectified again in order to supply the final consumer. Therefore, there is a significant reduction of cost, size and weight of an uninterruptible power supply function.

Referring to FIG. 2, it is seen that consumer 42 in FIG. 2 requires a large number of additional upstream components to provide a high voltage source which provides the peak power of the system. In comparison, the use of the architecture of FIG. 4 or FIG. 5 means that the provision of inrush current limitation and rectification can be reduced, thus saving cost, space and weight. The permanent connection of the electrical energy storage element 76 means that switching events (interruptions in the supplied power) are reduced are minimized in a transition between an operating mode and a backup mode.

A high-level approach to considering the operation of the system illustrated in FIG. 5 is to consider that it may operate in at least (i) a charging mode, (ii) an operating mode, and (iii) a backup mode. In the charging mode, the mains switch 90 connects the filter 92 and the charger 94 to the electrical energy storage element 96, but the contactor 98 is open, meaning that medical devices 100 are not powered.

In the operating mode, the circuit remains in the same state as the charging mode, with the alteration that the contactor circuit 98 is closed, enabling electrical energy to be supplied to the medical system 100, and also simultaneously enabling charging of the electrical energy storage element 96.

In the backup mode, the electrical energy storage element 96 may supply energy to the medical system 100 exclusively through the contactor circuit 98, in a situation where electrical energy is not received from the hospital utility mains supply 84, for example in a power loss situation.

Bypass switch 104 enables the electrical energy storage element 96 to be switched out of the supply route to the medical system 100. In a situation (not shown) where the bypass switch 104 is open, electrical energy is provided to the medical system 100 exclusively from the charging unit 94.

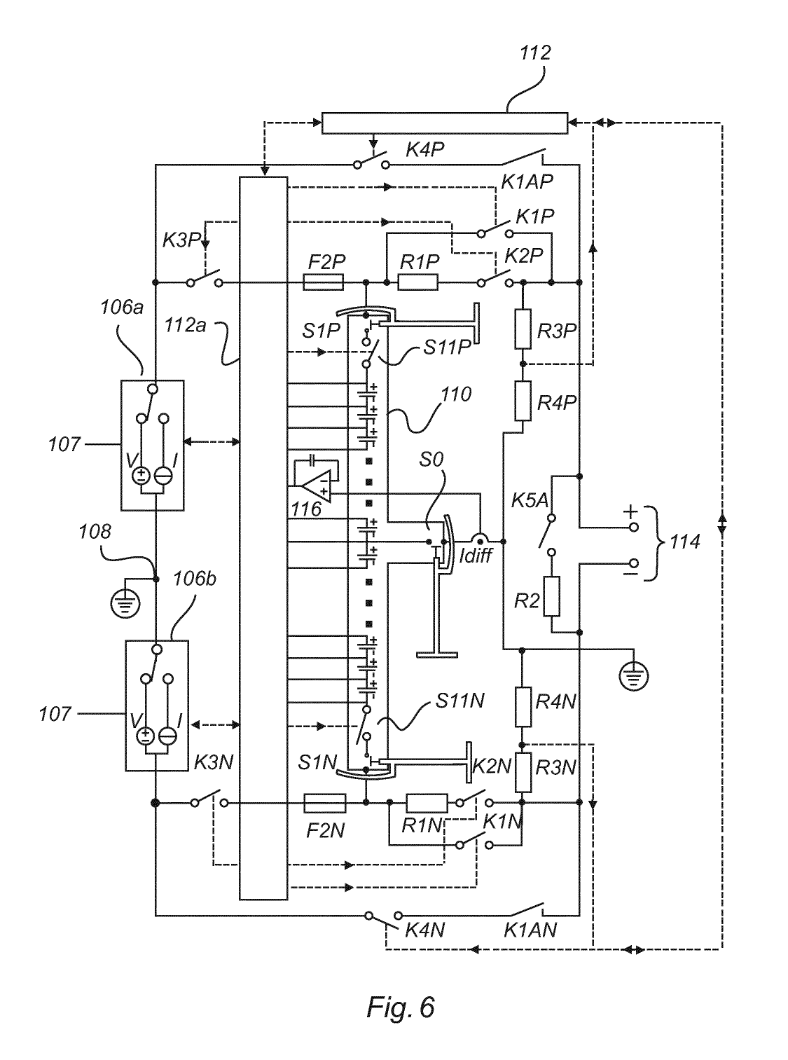

FIG. 6 shows a circuit schematic of a multifunctional power distribution apparatus according to the first aspect.

In FIG. 6, input terminals 107, a positive-side charging unit 106a and a negative-side charging unit 106b, an electrical energy storage element 110, DC load terminals 114, and a control unit 112 are provided. Also shown is a battery management system 112a which may be considered to be an extension of the control unit 112. A power switching network comprising switching means K1P, K1AP, K2P, K3P, K4P, and, K1N, K1AN, K2N, K3N, K4N, and K5A is provided. The switching means designation K1P versus K1N indicates a switching means having the same function, but being located in the positive or negative side of the circuit, respectively.

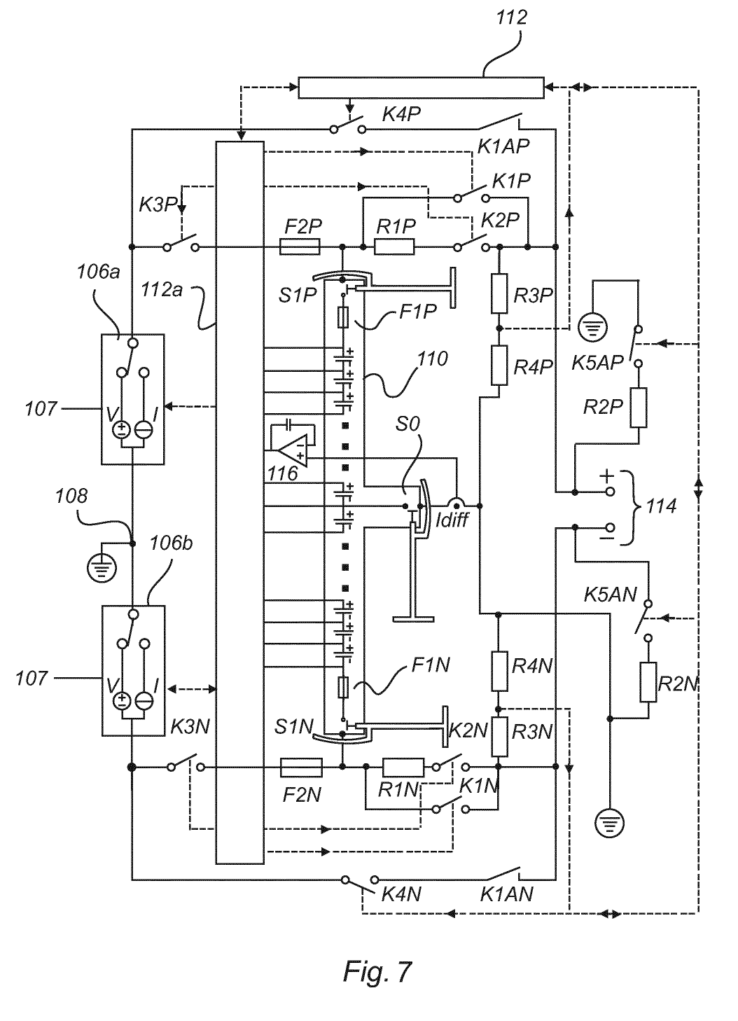

In an alternative embodiment, shown in FIG. 7, the switching means K5A and resistor R2 across the DC load terminals is replaced by the series connection of the positive side of the DC load terminal to protective earth via switching means K5AP and R2P, and by the series connection of the negative side of the DC load terminal to protective earth via switching means K5AN and R2N. In the subsequent description, it will be appreciated that when an event refers to K5A (of FIG. 6) undertaking a switching event, this is analogous to K5AP and K5AN being switched to the same position in unison.

In FIG. 7, the electrical energy storage element 110 comprises series fuses F1P and F1N, as an alternative to the contactors S11P and S11N of FIG. 6. These protect the electrical energy storage element 110 against an over-voltage. However, contactors could also be used for this purpose, as shown in FIG. 6.

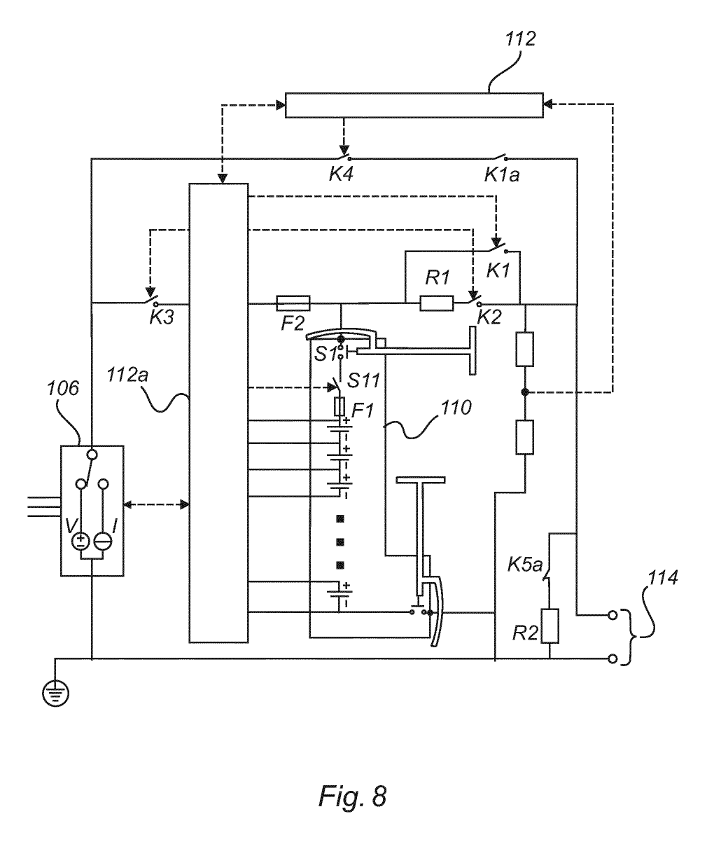

In FIG. 6, dotted lines represent control lines, and solid lines represent power-carrying lines. FIG. 6 shows a dual-rail multifunctional power distribution apparatus, although it would be appreciated that the principles discussed in relation to the embodiment of FIG. 6 may also be applied to a single-rail multifunctional power distribution apparatus as shown in FIG. 8.

In FIG. 6, there is shown a charging unit which is divided between a positive-side charging unit 106a and a negative-side charging unit 106b. The charging units 106a and 106b are connectable in use to the utility mains of a hospital, for example supplying 3-phase power. A connection to protective earth 108 between the charging units 106 is made 108.

An electrical energy storage element 110 is provided, which optionally may contain super capacitors, or a stack of battery cells capable of storing electrical energy. A control unit 112 is provided to control the power distribution apparatus, and a subset of the control unit 112 may be considered as a battery managing system 112a (BMS). The battery management system 112a has the function of monitoring the health of individual cells or subsets of small numbers of cells inside the electrical energy storage element 110. Such a battery management system may also be applicable to the monitoring of super capacitor stacks.

Electrical energy is supplied to a medical system via the DC load terminals 114. The connection of the power switching network in-between the charging units 106a and 106b, and the DC load terminals 114, in order to achieve the required functionality will now be discussed.

The positive-side charging unit 106a is connected to the positive terminal of the electrical energy storage element 110 via the switching means K3P and optionally the fuse F2P. The positive-side of the electrical energy storage element 110 is also connectable to the DC load terminals 114 via the switching means K1P. Similarly, the negative-side charging unit 106b is connectable to the negative-side of the electrical energy storage element 110 via switching means K3N, and optionally fuse F2N. The switching means K1N connects the negative-side of the electrical energy storage element 110 to the negative DC terminal 114.

The positive-side charging unit 106a is connectable directly to the positive DC terminal 114 via the switching means K4P, which forms a bypass path of the positive rail avoiding a connection to the electrical energy storage element 110. Optionally, a circuit breaker K1AP is provided in the positive bypass path. Similarly, the negative-side charging unit 106b is connectable directly to the negative terminal of the DC load terminals 114 via switching means K4N, and optionally circuit breaker K1AN.

The control unit 112 is connected (shown using the dotted lines) to control terminals of the switching means in the power distribution apparatus. The control unit 112 is connected to the battery management system 112a using a bidirectional communication means to enable feedback about the condition of the batteries to be given. Effectively, control unit 112 may be considered as an extension of the battery management system 112a.

Unidirectional control lines from the control unit are also provided to switching means K3P and K3N, to the bypass switching means K4P and K4N, and to the DC circuit switching means K1P and K1N, for example.

Resistors R3P and R4P are connected in series between the positive DC load terminal and the protective earth. Resistors R3N and R4N are connected in series between the negative DC load terminal and the protective earth. These series pairs of resistors form potential dividers for the positive and negative-side, respectively. The junction of the respective potential dividers is used as DC output voltage feedback signals, which are fed back to the control unit which may be connected to protective earth potential by its ground reference potential.

Another optional feature of the circuit of FIG. 6 is a transient switching arrangement, comprising resistor R1P and switching means K2P on the positive-side, and resistor R1N and switching means K2N on the negative-side. When medical equipment is switched into the power circuit for the first time, large capacitors may cause a significant inrush current. With no provision for this, damage to the charging units 106a, 106b and/or the electrical energy storage element 110 could occur. Therefore, R1P, K2P, R1N, and K2N are switched into the power supply path between the charger and/or the electrical energy storage element 110, and the DC load terminals, during transition states of the power switching network. This occurs moments before the main switching means K1P and K1P are switched into the path between the electrical energy storage element 110 and the DC load terminals 114.

Optionally, the resistors R1P and R1N may be replaced or supplemented by inductances, or resistive devices, which are designed to change their impedance dependent on their temperature. These kinds of components provide a significant positive or negative temperature coefficients (PTCs or NTCs).

Electrical energy storage element 110 is illustrated in FIG. 6 as being comprised of a series stack of battery cells. Alternatively, the electrical energy storage element 110 could be comprised of a series stack of super capacitors or a set of electrolytic or foil capacitors which may be comprise at least two single devices which are connected in series or parallel.

Optionally, the electrical energy storage element 110 is provided with a series switching means S11P and S11N. Optionally, the electrical energy storage element 110 is provided with a series fuse, or a switching device which is controllable from the battery management system 112a. Switching means S11P and S11N prevent discharge of the electrical energy storage element 110 during a fault condition, detectable by the control unit 112 or the battery management system 112a, for example.

In operation, the circuit shown in FIG. 6 has four principle states being (i) a charging mode, (ii) an operating mode, (iii) a backup mode, and (iv) a bypass mode.

Four subsidiary states forming transitions between the three principle states are also available. Table 1 illustrates the operation modes of the circuit, and the states of switching means K1, K1A, K2, K3, K4, and K5A. In dual-rail embodiments, the positive and negative switching means (denoted by the suffix -P or -N, respectively) are moved in unison. The table entry “0” indicates that the switching means connection is broken, or high-impedance. The table entry “1” indicates that the switching means connection is made, or low-impedance. In the following, the term “open” in relation to a switching means denotes a high-impedance path (substantially infinity Ohms). The term “closed” in relation to a switching means denotes a low-impedance path (substantially zero Ohms).

TABLE 1
Switching modes of the power switching network

| Operation Mode | K3 | K1 | K1A | K4 | K2 | K5A |
|---|---|---|---|---|---|---|
| ERROR | 0 | 0 | 1 | 0 | 0 | 0 |
| BACKUP OFF/ | 0 | 0 | 1 | 0 | 0 | 1 |
| Service | | | | | | |
| (i) CHARGING | 1 | 0 | 1 | 0 | 0 | 1 |
| OPERATING->Delay | 1 | 0 | 1 | 0 | 1 | 0 |
| (ii) OPERATING | 1 | 1 | 0 | 0 | 1 | 0 |
| OPERATING -> Delay -> | 0 | 0 | 1 | 0 | 1 | 0 |
| BACKUP | | | | | | |
| (iii) BACKUP | 0 | 1 | 0 | 0 | 1 | 0 |
| (iv) BYPASS | 0 | 0 | 1 | 1 | 0 | 0 |

In a charging mode (i) in which the electrical energy storage element is charged by the charging unit, power is not supplied to the DC load terminals 114, and a medical system connected to the DC load terminals 114 will be turned off. In the charging mode (i), switching means K3 and K3N are closed, to enable electrical energy to flow from the charging units 106a and 106b into the positive and negative-side of electrical energy storage element 110, respectively. At an appropriate stage of charge of the electrical energy storage element 110, the power distribution apparatus reconfigures the power switching network under the control of the control unit 112 from the charging mode (i) into the operating mode (ii), for example.

The system then transitions into the operating mode (ii), in which electrical energy is supplied to the DC load terminals 114 from the electrical energy storage element 110 and the charging units 106a and 106b, and the electrical energy storage element 110 can be charged. In this state, switching means K3N and K3P, switching means K1N and K1P, and optionally switching means K2P and K2N are closed, enabling charge to flow from the charging unit 106a, 106b to the positive and negative DC load terminals 114, respectively. In this mode, the electrical energy storage element 110 is also being charged.

If the control unit 112 detects a need to switch into a backup mode (for example, because mains power is lost), the control unit reconfigures the power switching network into a backup mode (iii) by opening switching means K3P and K3N, leaving K1P and K1N closed, K1AP and K1AN open, retaining K2P and K2N in their present state, and leaving K5P and K5N open. In this mode, electrical energy is supplied to the DC load terminals 114 exclusively from the electrical energy storage element 110. Thus, the transition from operating mode (ii) to backup mode (iii) is achieved by not affecting the main power path between the storage element 110 and the DC load terminals 114.

The multifunctional power distribution apparatus is also configurable into a bypass mode (iv) in which electrical energy is provided to the DC load terminals exclusively from the positive charging unit 106a, and the negative charging unit 106b. In the bypass mode, switching means K3P and K3N are open, switching means K1P and K1N are open, switching means K1AP and K1AN are closed, switching means K4P and K4N are closed, switching means K2P and K2N are open, and switching means K5A is open. Thus, the charging units on the positive-side and negative-side 106a and 106b supply electrical energy directly to the DC load terminals 114.

In the bypass mode, the bypass circuit is activated by closing the contacts K4 (on the positive and negative-side), while all contactors K1 to K3 and K5 are kept open. The bypass may be activated in case of failures of either the electrical energy storage element 110 or of the battery management section of the controller 112, 112a, because in this case the electrical energy storage element is isolated from the charging unit 106.

Table 1 also details a number of optional transitional modes.

Optionally, when transitioning from the charging mode (i) to the operating mode (ii), switching means K3 and switching means K1A on the positive and negative-side remain closed, and the switching means K2 on the negative-side and positive-side are closed.

In this case, the resistors R1P and R1N, presenting a medium-impedance path, are connected into the path of the DC load terminals 114 before the low impedance connection via the closed switching means K1P and K1N. This enables a DC-link of a connected medical system to be charged without a significant inrush current occurring. Such an inrush current could cause damage to the electrical energy storage element 110 or the charging unit 106 or any of the contactors K1P or K1N. This first transitional mode is represented in the “OPERATING->Delay” row of the table. The transitional step described previously for charging the DC-link of the medical system is suitable if large capacitances are present in the medical system connected to the DC load terminals 114.

Optionally, a terminal mode may be provided in which switching means K5a is closed. This enables a discharge of the input capacitor of a connected medical system. Such a mode is useful as a safety feature upon power-down of the medical system, for example.

Following the structural and functional description of the operation of the multifunctional power distribution apparatus of FIG. 6, variants will be discussed.

Optionally, extra fuses (not illustrated) are connected in series with the positive and negative sides of the electrical energy storage element 110, respectively. Such series fuses provide a failsafe current limit in the case of a battery fault condition. The fuses would be inserted into the circuit in place of, or in series with, S11P and S11N.

Optionally, mechanical service locks S1P and S1N are located between the fuses F1P and F1N. Optionally, another mechanical service lock S0 may be placed in order to completely disconnect the battery centre tap from protective earth. Such mechanical or logically interconnected service locks allow access to terminals of the electrical energy storage element 110 only if the electrical contacts of the electrical energy storage element 110 are disconnected. The mechanical service locks are interconnected, such that touching a terminal is only possible if all electrical connections between the electrical energy storage element and the terminals are open.

Optionally, breaker K5a is connected across the DC load terminals 114 in series with resistance R2. This forms a discharge circuit between the DC terminals of the DC power bus which can discharge electrical energy held in capacitances in connected items of consumer equipment. Another embodiment of the discharge circuit may consist of a series connection of K5AP and R2P connected between the positive potential of the DC load terminal 114 and protective earth, and K5AN and R2N which are connected between P.E. and the negative DC load terminal 114, as illustrated in FIG. 6.

Optionally, a current integration circuit 116 is connected between the battery managing system 112a and the protective earth. This circuit integrates the differential current I_{diff}, shown in FIG. 6, to enable a battery fault condition to be detected. Therefore, equal charging or discharge currents in both battery portions can be provided by readjustment of the set-points for the current sources 106a and 106b.

The control unit 112, 112a may be implemented using a microprocessor, a microcontroller, an FPGA, or another digital processing system. Logic interfaces to the switching means may be made using custom communication systems, or a MODBUS™ or FIELDBUS™ system, for example.

According to an embodiment, the charging unit 106 of the multifunctional power distribution apparatus is configured to supply an average power drawn by a medical imaging apparatus to the electrical energy storage element 110 of the multifunctional power distribution apparatus.

The electrical energy storage element 110 is preferably comprised of a battery, such as a lithium ion cell stack, or a super capacitor. In FIG. 6, the entire stack is composed of two partial stacks which are connected in series, and which provide a centre tap terminal in order to connect the electrical energy storage element 110 to a protective earth 108.

Optionally, a DC fuse is connected between the outer cell of the electrical energy storage element 110 and the power terminals of the electrical energy storage element. This serves as a disconnector in the case of a short-circuit. Optionally, contactors S11P and S11N may be replaced or supplemented by DC-fuses.

Optionally, the mechanical service locks S1P, S1N completely disconnects the battery terminals, in case of removal of the casing of the battery for example.

Optionally, a current sensor configured to monitor a differential current flowing between the electrical energy storage element 110 and the protective earth node is provided by the integrator 116, ensuring equal charge flows from both sides of the electrical energy storage element 110.

The connection of the two electrical energy storage element 110 halves to protective earth 108 implies that the positive charging unit 106a and the negative charging unit 106b may provide an unequal charge. Unequal states of charge of the halves of the electrical energy storage element 110 are undesired because in this case, the state of charge of the entire element is reduced to the state of charge of the half in which the charge is lower. At a certain state of charge, the voltage across this half may drop due to low state of charge whereas the complementary half is at a high level of charge. This may lead to unequal voltage across the two poles of the electrical energy storage element 110. This effect may occur if the actual current provided by the positive charging unit 106a and the negative charging unit 106b differ from each other. After several cycles of charge and discharge, a state may occur that one of the halves is completely charged whereas the complementary half is almost completely discharged. In this case, the performance of the battery is significantly reduced and accelerated ageing may be the consequence.

Therefore, the control unit 112 can be configured to compensate for this difference in charge actively. The battery management system 112a may be configured to calculate a first current set point used for the positive half of the electrical energy storage element 110, and a second current set point used for the negative half of the electrical energy storage element 110. The integrator 116 may be configured to integrate the current difference signal I_{diff} of FIG. 6 for calculating the first current set point and the second current set point.

The battery management system may be configured to be operated as an integral controller or as a proportional-integral controller or as a proportional-integral-derivative controller to correct the charge level of the positive and negative portions of the electrical energy storage element 110.

Optionally, the DC fuse F2P and the DC fuse F2N provide safety link between the charging units 106a, 106b, and the electrical energy storage element 110, in case of an over current due either to a fault across the DC load terminals, or in the electrical energy storage element 110. These DC fuses are dimensioned according to the maximum charging current required by the charging unit 106a, and 106b.

Optionally, the battery management system 112a is configured to supervise the voltage across a plurality of the cells of the electrical energy storage element 110. The battery management system 112a detects and indicates failures and imbalances between the voltages across any of cells or across pluralities of a few cells. For example, the battery management system 112a may employ active balancing or passive balancing techniques to ensure an appropriate voltage balance across the cells.

Optionally, the power distribution apparatus is configured to detect a current level of battery charge inside the battery management system 112a. When functioning in the backup mode, an indication of the current charge level is measured. Optionally a prediction of the remaining operating time of equipment connected to the DC load terminals can be provided to a user. Therefore, in a fault condition of the utility power source, a medical professional may be provided with an estimate of how much time is remaining to finish an operation.

Optionally, an interlock is provided enabling the connections between the electrical energy storage element 110 and the consumers only if the discharge unit is disconnected by the contact K5A. The interlock can be implemented in the switching devices K1 to K5, or within the control unit.

According to the above described solution, in the case of a significant mains fault, or a total mains breakdown, the consuming circuits would hardly be affected. The architecture inherently comprises a backup function, enabling connected systems to remain operational. The charging unit 106 decouples the energy storage element 110 as well as the consuming circuits completely from the supplying utility mains. In addition, the electrical energy storage element 110 may be dimensioned to supply the system during normal operation up to the consumed peak power level, so that the buffer can proceed to supply the system in backup mode without a performance reduction, until the entire stored energy in the electrical energy storage element 110 is depleted. This is advantageous in the case of a loss of utility power during an interventional operation with a patient. In addition, the transition between the operating mode (ii) and the backup mode (iii) may be achieved without an interruption in the supply voltage, because the electrical energy storage element is always connected, in this transition.

In a system which comprises one or more consumers configured to draw pulses of a very high peak power, with a small duty cycle, the energy for this peak power level is transmitted from the electrical energy storage element 110 to the consumer only via wires, fuses, closed contactors or breakers, (and optionally filters). Therefore, power converters rated for the peak power level are not needed in the path to supply such consumers, saving component costs.

Fluctuations of power consumption of the system can be buffered and balanced by the electrical energy storage unit 110. The electrical energy storage unit 110 can supply the system with its peak power requirement, whereas it is charged continuously at a much lower power level. The room installation parts for the incoming utility power only need to be dimensioned to the lower power level, which equates to the level of maximum average power consumption. Therefore, installation effort and expense can be reduced.

As an example, a C-arm system, or a CT scanner, may be considered. The short-term peak power of such systems may be on the order of magnitude up to 150 kW, whereas the average power may be on the order of magnitude of 10 kW. If the pulse energy is buffered by a battery, both the hospital utility mains installation, and the charging unit of the system, can be dimensioned for 10 kW, and not for 150 kW. The hospital utility mains system is also not stressed by large and sudden peak power pulses. This avoids corresponding dips in the mains voltage of a hospital, and reduces immunity requirements required for other systems which are supplied from the same mains.

According to an alternative embodiment, a power distribution apparatus as described above and illustrated in FIG. 6 can be provided, wherein one charger 106 is connected to the positive and negative rails using voltage limiting circuits. In this case, only one charging unit is needed.

It will be appreciated that it is possible to provide a single rail version of the circuit, in which the battery stack or super capacitor stack is connected between the protective earth and only one positive rail.

FIG. 8 shows a cost-saving implementation which can be provided by omitting one half of the battery stack and a corresponding half of a battery management system 112a. In the case of FIG. 8, the control unit 112, the electrical energy storage element 110, the charging unit 106, and the DC load terminals 114 are provided as discussed previously in connection with FIG. 6. A difference between the implementation of FIG. 6 and FIG. 8 is that the negative rail set of switches, fuses, wires and control means are omitted. This implementation is advantageous at a lower level of the DC buffer voltage. The previously mentioned advantage of a common charger for average power, and the ability to de-rate components upstream of the charger remain.

According to a third aspect, there is provided a method for controlling a multifunctional power distribution apparatus.

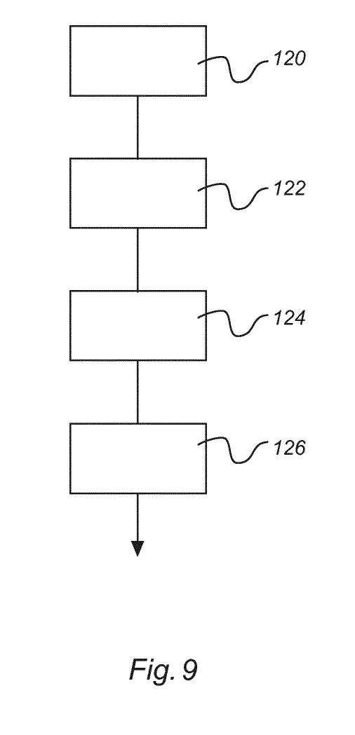

FIG. 9 shows a method according to the third aspect.

The method comprises the steps of:
a) charging the electrical energy storage element using the charging unit;
b) monitoring, using the control unit of the multifunctional power distribution apparatus, a power demand requirement of a load connected to the DC load terminals of the multifunctional power distribution apparatus using the control unit;
c) computing a configuration of the power switching network using the power demand requirement of the load;
d) configuring the power switching network into one of (i) a charging mode, (ii) an operating mode, (iii) a backup mode, and (iv) a backup mode.

According to an embodiment of the third aspect, there is provided a method further comprising the steps of:

a1) detecting a fault condition of the source of electrical energy at the input terminals;
d1) configuring the power switching network into the backup mode;
further comprising step e):
e) supplying electrical energy to the load exclusively from the electrical energy storage element.
According to a second aspect, there is provided a medical equipment system 15.
FIG. 1 illustrates an example of a medical equipment system.

The medical equipment system 15 comprises:

a medical imaging apparatus 10; and

the multifunctional power distribution apparatus as described above.

The input terminals of the multifunctional power distribution apparatus are connectable to a utility power supply, and the DC load terminals of the multifunctional power distribution apparatus is configured to supply electrical energy to the medical imaging apparatus 10. The charging unit of the multifunctional power distribution apparatus is configured to supply an average power drawn by the medical imaging apparatus to the electrical energy storage element of the multifunctional power distribution apparatus.

According to a fourth aspect of the invention, a computer program element for controlling an apparatus according to one of the first aspect or its embodiments or variations is provided, which, when the computer program element is executed by a control unit, is adapted to perform the steps of one of the third aspect, or its embodiments.

According to a fifth aspect of the invention, there is provided a computer-readable medium having stored the computer program element of the fourth aspect.

A computer program element might therefore be stored on a computer unit, which might also be part of an embodiment of the present invention. This computing unit may be adapted to perform or induce performance of the steps of the method described above.

Moreover, it may be adapted to operate the components of the above described apparatus. The computing unit can be adapted to operate automatically and/or to execute the orders of a user. A computer program may be loaded into a working memory of a data processor or of any kind of programmable logic device or programmable gate-array. The data processor may thus be equipped to carry out the method of the invention.

This exemplary embodiment of the invention covers both the computer program that has the invention installed from the beginning, and a computer program that by means of an update turns an existing program into a program that uses the invention. A computer program may be stored and/or distributed on a suitable medium, such as an optical storage media or a solid state medium supplied together with, or as a part of other hardware, but may also be distributed in other forms, such as via the Internet or other wired or wireless telecommunication systems.

However, the program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It should to be noted that embodiments of the invention are described with reference to different subject-matters. In particular, some embodiments are described with reference to method-type claims, whereas other embodiments are described with reference to the device-type claims. However, a person skilled in the art will gather from the above, and the following description, that unless otherwise notified, in addition to any combination of features belonging to one type of subject-matter, also any other combination between features relating to different subject-matters is considered to be disclosed with this application.

All features can be combined to provide a synergetic effect that is more than the simple summation of the features.

While the invention has been illustrated and described in detail in the drawings and foregoing description, such illustration and description are to be considered illustrative or exemplary, and not restrictive. The invention is not limited to the disclosed embodiments.

Other variations to the disclosed embodiments can be understood, and effected by those skilled in the art in practicing the claimed invention, from a study of the drawings, the disclosure, and the dependent claims.

In the claims, the word “comprising” does not exclude other elements or steps, and the indefinite article “a” or “an” does not exclude a plurality. A single processor, or other unit, may fulfil the functions of several items recited in the claims. The mere fact that certain measures are recited in mutually different dependent claims does not indicate that a combination of these measures cannot be used to advantage. Any reference signs in the claims should not be construed as limiting the scope.

1. A multifunctional power distribution apparatus, comprising:
input terminals enabling connection of the apparatus to a source of electrical energy;
a charging unit connected to the input terminals;
an electrical energy storage element configured to receive electrical energy from the charging unit;
DC load terminals configured to supply electrical energy to a load;
a power switching network enabling an adaptable configuration of the charging unit, the electrical energy storage element, and the DC load terminals; and
a control unit configured to control the charging unit and the power switching network;
wherein the control unit is configured to set the power switching network into at least one of the following modes: (i) a charging mode in which the electrical energy storage element is charged by the charging unit, (ii) an operating mode in which electrical energy is supplied to the DC load terminals from the electrical energy storage element and the charging unit, and the electrical energy storage element can be charged, (iii) a backup mode in which electrical energy is supplied to the DC load terminals exclusively from the electrical energy storage element, and (iv) a bypass mode in which electrical energy is provided to the DC load terminals exclusively from the charging unit.

2. The power distribution apparatus according to claim 1, wherein the charging unit is configured to charge the electrical energy storage element using at least one of: an adjustable DC current, an adjustable DC voltage, according to a predefined charging curve, and according to a predefined charging characteristic.

3. The power distribution apparatus according to claim 1, wherein the electrical energy storage element comprises a positive-side electrical energy storage element and a negative-side electrical energy storage element storage element both connected to a protective earth node.

4. The power distribution apparatus according to claim 3, further comprising:
a current sensor configured to monitor a differential current flowing between the electrical energy storage element and the protective earth node, wherein the control unit is configured to adjust a set point of the charging unit in order to minimize the differential current between the positive and negative side electrical energy storage elements.

5. The power distribution apparatus according to claim 1, further comprising:
an electrical energy storage element management system;
wherein the electrical energy storage element comprises a plurality of cells; and
wherein the electrical energy storage element management system is configured to supervise cells of the plurality of cells of the electrical energy storage element, to detect an undesired state between cells of the electrical energy storage element, and to compensate for the undesired state.

6. The power distribution apparatus according to claim 1, wherein the charging unit is configured to provide an average power level of an expected load characteristic at the charging unit output terminals.

7. The power distribution apparatus according to claim 1, wherein the control unit is further configured to set the power switching network into a transition mode between the charging mode and the operation mode; wherein in the transition mode, the power switching network is configured to connect a series resistor between the electrical energy storage element and the DC load terminals, to prevent the occurrence of an inrush current.

8. The power distribution apparatus according to claim 1, further comprising:
a charge level detector configured to obtain a charge level of the electrical energy storage element;
wherein the control unit is further configured to compute a remaining operating time of equipment connected to the multifunctional power distribution apparatus based on the charge level of the electrical energy storage element.

9. The power distribution apparatus according to claim 1, wherein the power switching network comprises a first switching element configurable to connect the electrical energy storage element to the DC load terminals; a second switching element configurable to connect the output of the charging unit to the electrical energy storage element; and a third switching element configurable to connect the output of the charging unit directly to the DC load terminals.

10. The power distribution apparatus according to claim 1, further configured to prevent the occurrence of a switching event in the path between the electrical energy storage element and the DC load terminals during a transition between the operating mode and the backup mode.

11. A medical equipment system, comprising:
a medical imaging apparatus; and
a multifunctional power distribution apparatus comprising:
input terminals enabling connection of the apparatus to a source of electrical energy;
a charging unit connected to the input terminals;
an electrical energy storage element configured to receive electrical energy from the charging unit;
DC load terminals configured to supply electrical energy to a load;
a power switching network enabling an adaptable configuration of the charging unit, the electrical energy storage element, and the DC load terminals; and
a control unit configured to control the charging unit and the power switching network;
wherein the control unit is configured to set the power switching network into at least one of the following modes: (i) a charging mode in which the electrical energy storage element is charged by the charging unit, (ii) an operating mode in which electrical energy is supplied to the DC load terminals from the electrical energy storage element and the charging unit, and the electrical energy storage element can be charged, (iii) a backup mode in which electrical energy is supplied to the DC load terminals exclusively from the electrical energy storage element, and (iv) a bypass mode in which electrical energy is provided to the DC load terminals exclusively from the charging unit;
wherein the input terminals of the multifunctional power distribution apparatus are connectable to a utility power supply, and the DC load terminals of the multifunctional power distribution apparatus are configured to supply electrical energy to the medical imaging apparatus.

12. A method for controlling a multifunctional power distribution apparatus, comprising:
charging an electrical energy storage element using a charging unit;
monitoring, using a control unit of the multifunctional power distribution apparatus, a power demand requirement of a load connected to DC load terminals of the multifunctional power distribution apparatus;
computing a configuration of a power switching network using a power demand requirement of a load; and
configuring the power switching network into at least one of (i) a charging mode, (ii) an operating mode, (iii) a backup mode and (iv) a bypass mode.

13. The method according to claim 12, further comprising:
detecting a fault condition of the source of electrical energy at the input terminals;
configuring the power switching network into the backup mode; and
supplying electrical energy to the load exclusively from the electrical energy storage element.

14. (canceled)

15. A non-transitory computer readable medium having one or more executable instructions stored thereon, which when executed by a processor, cause the processor to perform a method for controlling a multifunctional power distribution apparatus, the method comprising:
charging an electrical energy storage element using a charging unit;
monitoring, using a control unit of the multifunctional power distribution apparatus, a power demand requirement of a load connected to DC load terminals of the multifunctional power distribution apparatus;
computing a configuration of a power switching network using a power demand requirement of a load; and
configuring the power switching network into at least one of (i) a charging mode, (ii) an operating mode, (iii) a backup mode and (iv) a bypass mode.
